(19) United States
(12) Patent Application Publication
(10) Pub. No.: US 2008/0195125 A1
(43) Pub. Date: Aug. 14, 2008

(54) DEVICE FOR HEART BYPASS SURGERY AND ANASTOMOSIS

(76) Inventor: Grant T. Hoffman, Bloomington, IN (US)

Correspondence Address: BRINKS HOFER GILSON & LIONE/CHICAGO/COOK, PO BOX 10395, CHICAGO, IL, 60610, US

(21) Appl. No.: 12/029,295

(22) Filed: Feb. 11, 2008

Related U.S. Application Data
(60) Provisional application No. 60/900,819, filed on Feb. 12, 2007.

(51) Int. Cl. A61B 17/11

(52) U.S. Cl. 606/153

(57) ABSTRACT

Anastomoses devices including a stent structure or structures are provided. In addition, a method of using an anastomosis device to perform anastomoses quickly and efficiently is provided. One advantage of the devices and methods is that they may provide for quick and effective attachment to a vessel. A further advantage is that the second end portion of the anastomosis device may provide for anastomosis without causing trauma to vessel walls and other harmful consequences.

The present patent document claims the benefit of the filing date under 35 U.S.C. §119(e) of Provisional U.S. Patent Application Ser. No. 60/900,819, filed Feb. 12, 2007, which is hereby incorporated by reference.

### BACKGROUND

The present invention relates to medical devices and more particularly, to devices for anastomosis. The invention further provides methods of manufacturing devices for anastomosis and methods of treating vessel impairment with devices for anastomosis.

Anastomosis is the process of connecting two or more ends of a hollow tube to a vessel in order to provide an alternate channel for fluid flow. In humans, surgical anastomosis is performed to provide an alternate channel for the flow of bodily fluids.

Anastomosis is often an appropriate surgical procedure when body vessels become impaired due to a variety of factors. For example, blood vessels may be impaired by becoming clogged, blocked, narrowed, or otherwise impaired. When the blood vessels of the vascular system fail to function properly, severe health consequences can result, including death. Among the most serious forms of vascular impairment is coronary artery atherosclerosis. According to one estimate, close to 14 million Americans have coronary artery atherosclerosis. Annually, an estimated 1.5 million people develop the most severe type of coronary artery disease—acute myocardial infarction. Roughly a third of the people struck by acute myocardial infarction die as a result of this type of coronary artery disease.

It is estimated that in 2006 approximately 250,000 coronary bypass surgeries will be performed in the United States. It is common during coronary bypass surgery to perform as many as five anastomoses. Although coronary bypass surgery is an invasive procedure, it is often the only available treatment option. However, there are drawbacks associated with coronary bypass surgery and other types of bypass surgery. Complications can arise from bypass surgery including myocardial infarction, cardiac arrhythmias, infection, edema, thrombosis, blood clot formation, restenosis, nerve injury, and graft occlusion.

During traditional coronary bypass surgery, the sternum is cut down the middle with a bone saw and the chest is opened. The surgeon may elect to place the patient on cardiopulmonary bypass. In addition, the surgeon may use stabilizing devices to hold the heart still. After locating the impaired artery, a surgeon typically creates an incision in the artery on one side of the blood vessel impairment. Next, the surgeon sutures a graft to the artery with between eight and fourteen evenly-spaced sutures. After one end is attached, the surgeon creates an incision on the other side of the blood vessel impairment and the other end of the graft is sutured to the artery. Usually, a surgeon will check for leakages to ensure that the graft is securely in place and correctly aligned within the body. Finally, the surgeon closes the chest cavity after all the necessary anastomoses are complete.

One problem with bypass surgery is that the internal structures of the body are left exposed for an extended length of time. As the length of exposure increases, the risk of infection and other complications may increase. Not only is the risk to the patient increased by a lengthier procedure, the cost of such a procedure likewise increases. For example, more medications, such as anesthesia, are needed for longer procedures. Similarly, additional staff resources, such as staff time and equipment time, are required as the length of a procedure increases.

Another problem with traditional bypass surgery is that the skill of the surgeon may negatively affect the success of the bypass procedure. The skill of a surgeon in suturing a graft to existing arteries may determine whether leakages occur or result in other associated problems.

In addition to traditional methods of anastomoses, devices for anastomosis have been developed in attempts to address some of these problems. However, the available devices have had problems including occlusion, thrombus, stenosis at the connector site, aortic dissection associated with device deployment, graft kinking, and postoperative device detachment. Therefore, it is apparent to the inventor that an improved anastomosis device would be desirable.

### SUMMARY

An anastomosis device including a stent structure is described. The anastomosis device comprises a graft tube, a first end portion, and a second end portion. The graft tube comprises a first end, a second end, and a lumen extending therethrough. The first end portion is connected to the first end of the graft tube and the first end portion is adapted to be attached to an incision in a vessel. The second end portion includes a stent structure. Furthermore, the second end portion is connected to the second end of the graft tube and is adapted to be attached to another incision in the vessel. The second end portion comprises a first part adapted to be connected to the second end of the graft tube. The first part is substantially cylindrical. A second part is substantially quonset-shaped and the first part is connected to the convex region of the second part. Additional details and advantages are described below in the detailed description

A method of using an anastomosis device is also described. First, an incision is created in a vessel. Next, a first end portion of an anastomosis device is attached to the incision. A second incision is then created in a vessel and a compressed second end portion of an anastomosis device is inserted through the second incision. Next, the second end portion is expanded to a generally relaxed state and a graft tube is attached to the second end portion.

### BRIEF DESCRIPTION OF SEVERAL VIEWS OF THE DRAWINGS

The invention may be more fully understood by reading the following description in conjunction with the drawings, in which:

FIG. 1 is a perspective view of an anastomosis device;

FIG. 2A is a side view of one end of an anastomosis device and FIG. 2B is an end view of the end of the anastomosis device from FIG. 2A;

FIGS. 3A, 3B, 3C, 3D, and 3E are partial cut away views of a method of using an anastomosis device;

FIG. 4 is a perspective view of an anastomosis device; and

FIGS. 5A, 5C, 5E, 5G, and 5I are side views of ends of anastomosis devices and FIG. 5B, 5D, 5F, and 5H are end views of the ends of the anastomosis devices shown in FIG. 5A, 5C, 5E, 5G, and 5J, respectively.

### DETAILED DESCRIPTION

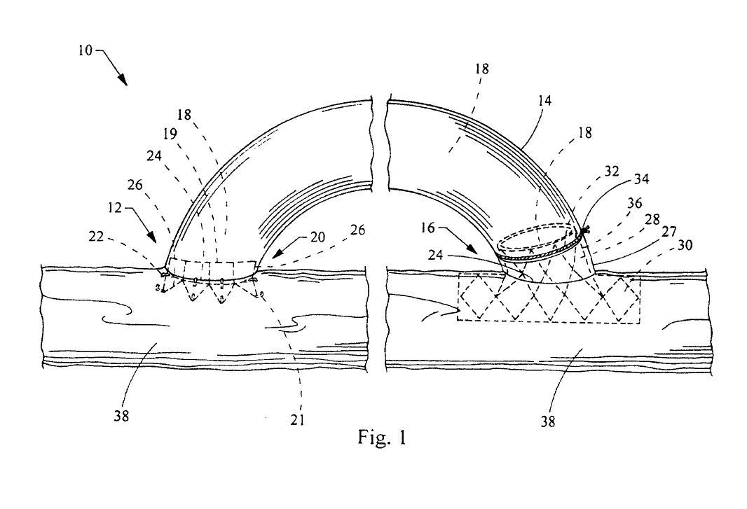

Referring to the drawings, and specifically to FIG. 1, an improved anastomosis device 10 is shown. The anastomosis device 10 includes a first end portion 12, a graft tube 14, a second end portion 16, and a lumen 18 running through the interior of the anastomosis device 10. The first end portion 12 in this embodiment includes a conventional device for securing a graft tube 14 to an incision 24 in a vessel 38 during anastomosis. The conventional device may be a prior art device as shown in U.S. Pat. No. 6,451,048, hereby incorporated by reference. As shown in FIG. 1, the first end 20 of the graft tube 14 is attached to the vessel 38 with a number of lower connector wires 21 and upper connector wires 22 attached to frame wires 19. The lower connector wires 21 and upper connector wires 22 secure the device to the vessel 38 by penetrating the vessel 38. Referring now to the second end portion 16, the second end portion 16 includes a stent structure 36 encapsulated in a polyurethane. The stent structure 36 includes a first part 28 and a second part 30. In this embodiment, the first part 28 is substantially cylindrical and the second part 30 extends laterally from the edge of the first part 28. A sewing ring 32 is disposed on the first part 28 in the region where the second end 27 of the graft tube 14 is external to the first part 28. A suture 34 is shown wrapped around the graft tube 14 over the sewing ring 32 to secure the graft tube 14 to the first part 28.

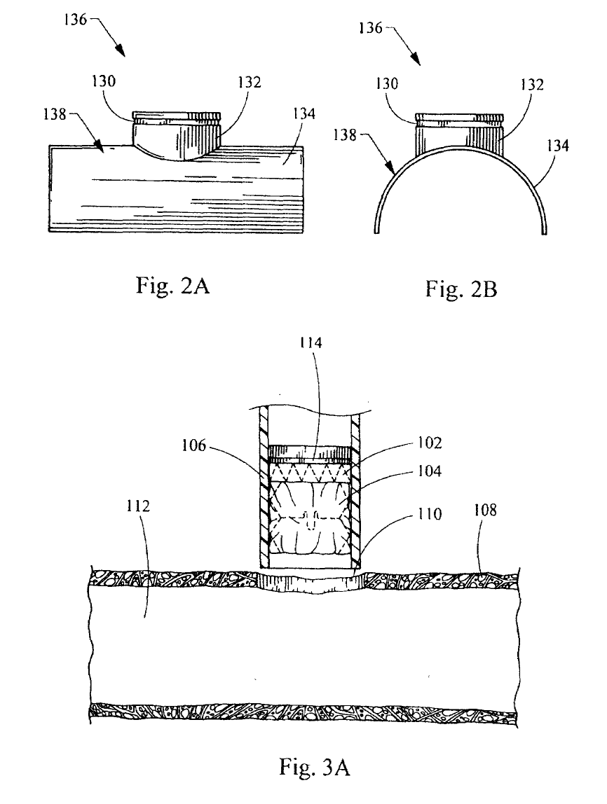

Referring to FIGS. 2A and 2B, the stent structure 136 is shown as a solid structure in FIGS. 2A and 2B for illustration. However, it is preferred that the stent structures be made from flexible struts as shown in FIG. 1. The stent structure 136 shown in FIGS. 2A and 2B is similar to the stent structure 36 shown in FIG. 1 with a substantial difference. In FIGS. 2A and 2B, the first part 132 of the stent structure 136 is substantially the same height on all sides with respect to the second part 134. On the other hand, the first part 28 of the stent structure 26 shown in FIG. 1 has a smaller height on the side of the stent structure facing towards the first end portion 12 than does the opposite side of the first part 28. It is preferred that the first part 28 has a smaller height on the side of the stent structure 26 facing towards the first end portion 12 as shown in FIG. 1. Referring again to FIG. 2A, a first part 132 is attached to the convex region 138 of an expanded second part 134. FIG. 2B shows an end view of a first part 132 attached to a second part 134 as seen in FIG. 2A. In addition, the first part 132 includes a sewing ring 130. The sewing ring 130 may be a recessed groove or other structure integrally formed into the first part 132 of the stent structure 136 that is adapted to receive a suture, or other connecting device, wrapped around the first part 132. In this case, the first part 132 is substantially cylindrical and the second part 134 extends laterally and at a generally right angle from the first part 132. The second part 134 is substantially quonset-shaped in this embodiment. When expanded, the second part 134, as in FIGS. 2A and 2B, may be quonset-shaped. In general, the term “quonset-shaped” typically refers to a shape that is one half of a right circular cylinder which has been divided by a plane passing through its axis of symmetry. However, according to embodiments that have a quonset-shaped part, the part is not necessarily entirely quonset-shaped. For example, the right circular cylinder need not be divided by a plane passing through the axis. The walls of the right circular cylinder may also extend further than or not as far as the axis of symmetry. Moreover, the quonset-shaped object may be more annular when the edges of the quonset are shortened in particular directions. Other geometries are also contemplated according to the invention.

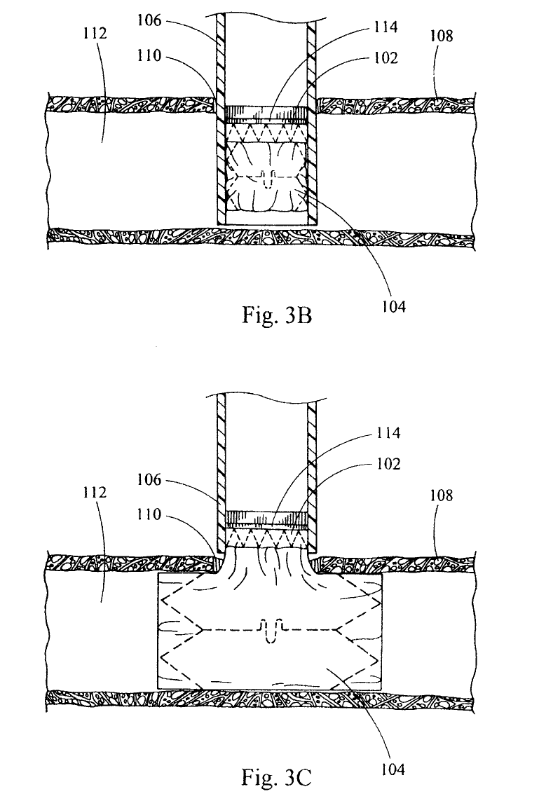

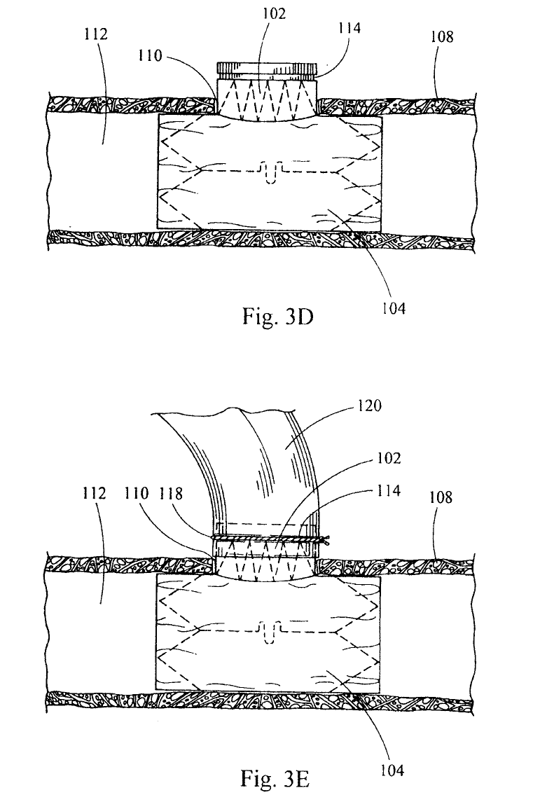

FIGS. 3A, 3B, 3C, 3D, and 3E show various stages illustrating a method of using an anastomosis device. As shown in FIG. 3A, an incision 110 has been made in a vessel 108. A first part 102 and second part 104 are shown fixed together and compressed within a retention sheath 106. A sewing ring 114 is disposed on the first part 102. The incision 110 is large enough to allow the retention sheath 106 to be inserted into the lumen 112 of the vessel 108. FIG. 3B shows the retention sheath 106 containing the first part 102 and the second part 104 inserted through the incision 110 and into the lumen 112 of the vessel 108. FIG. 3C shows the retention sheath 106 partially withdrawn, which allows the second part 104 to adopt a generally relaxed configuration within the lumen 112 of the vessel 108. As shown in FIG. 3C, the first part 102 remains compressed within the retention sheath 106. FIG. 3D shows the first part 102 and second part 104 expanded. Finally, FIG. 3E shows a graft tube 120 attached to a first part 102 with a suture 118 tied around a sewing ring 114. As shown in FIGS. 3C, 3D, and 3E, the expanded second part 104 may extend substantially into the vessel 108, thereby being adapted to engage substantially the entire circumference of a vessel wall. The second part 104 may also extend far enough along a vessel wall so that the first part 102 extends above the incision 110 when the second part rests on the vessel wall opposite the incision 110. When this is the case, the graft tube 120 may easily be attached to the first part 102. The first part 102 may also be attached to the graft tube 120 by applying pressure to the vessel wall opposite the incision to raise the first part 102 sufficiently above the incision 110 to attach the graft tube 120. In addition, the wall of the vessel surrounding the incision 110 may be depressed to position the first part 102 above the incision 110 to allow the graft tube 120 to be connected to the first part 102.

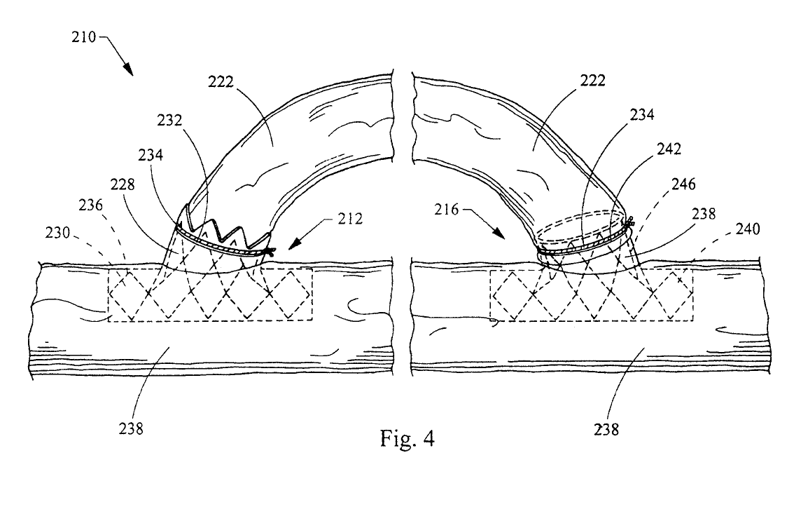

Referring now to FIG. 4, an anastomosis device 210 is shown with a first end portion 212 and a second end portion 216. Both the first end portion 212 and second end portion 216 include stent structures 236, 246 in this embodiment. The stent structure 236 of the first end portion 212 includes a first part 228 and a second part 230. A sewing ring 232 is shown disposed on the first part 228. The first part 228 may have various configurations, and here the top of the first part 228 is shown with a zig-zag shape. Similar to FIG. 1, the first part 228 of the first end portion 212 is configured to lean in the direction of the second end portion 216. Likewise, the first part 246 of the second end portion 216 is configured to lean in the direction of the first end portion 212. The stent structure 246 of the second end portion 216 includes a first part 246, a second part 240, and a sewing ring 242. The graft tube 222 in this case is a harvested vessel.

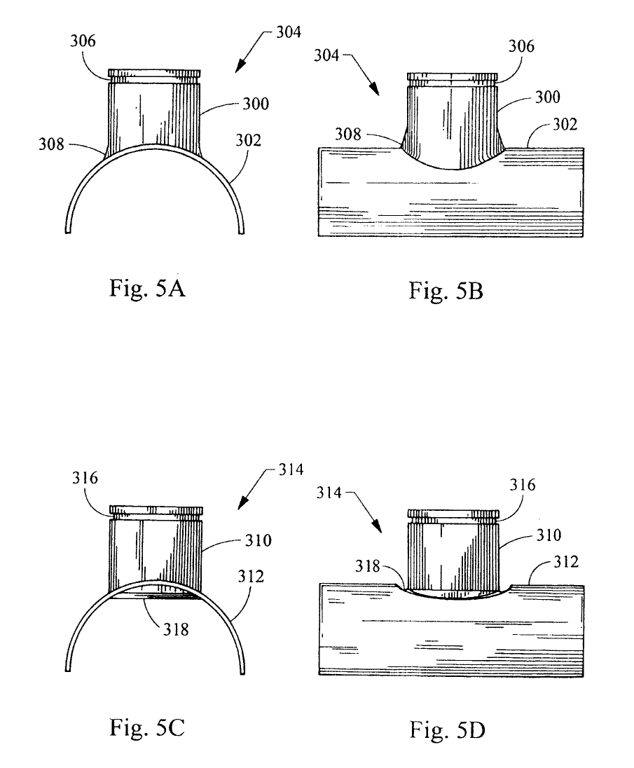

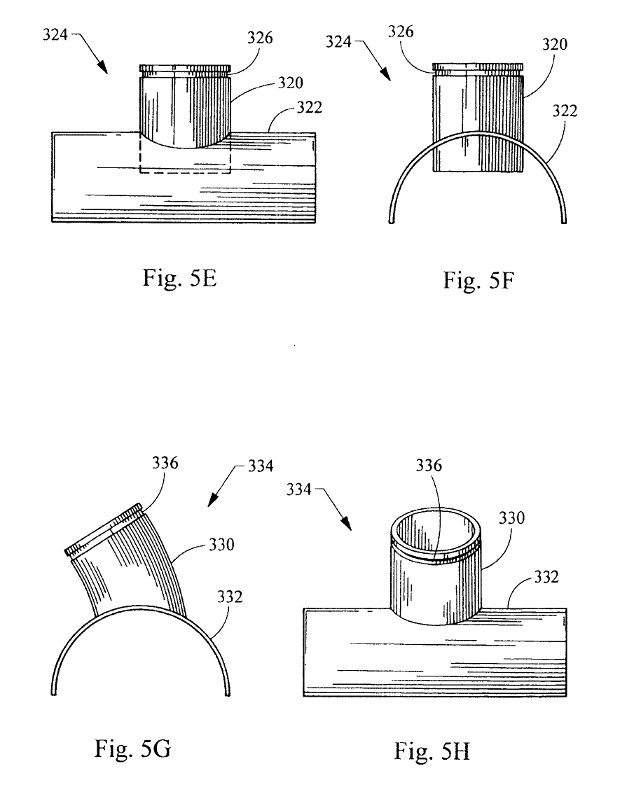

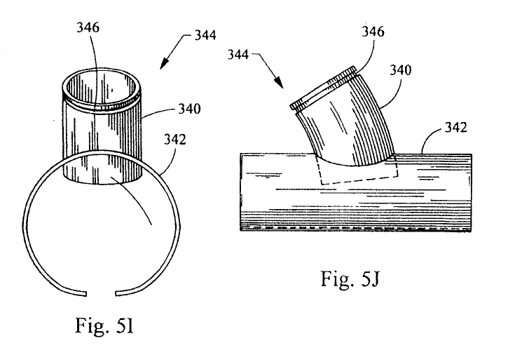

Referring now to FIGS. 5A and 5B, FIGS. 5A and 5B show a stent structure 304 with a sewing ring disposed on the first part 300. The second part 302 of the stent structure is connected to the second part 302. In this case, the first part 300 has a flaring portion 308 on the bottom where the first part connects to the second part 302. The second part 302 extends generally laterally from the first part 300. As seen in FIGS. 5A and 5B, this is one of the various geometries contemplated by quonset-shaped. As previously discussed and as shown in FIGS. 2A and 2B, a quonset-shaped second part 134 may intersect a first part 132 at substantially a right angle and curve downwardly away from the first part 132. Alternatively, as shown in FIGS. 5C and 5D, to form a quonset-shaped second part 312, the second part 312 of the stent structure 314 includes a recessed region 318 near where the first part 310 intersects the second part 312. In FIGS. 5E and 5F, the second part 322 of the stent structure 324 is connected to the first part 320 and a portion of the first part 320 extends below the intersection of the second part 322 with the first part 320. FIGS. 5E and 5F also include a sewing ring 326.

In FIGS. 5G and 5H, the stent structure 334 includes a first part 330 and a second part 332 extending generally laterally from the first part 330. FIGS. 5G and 5H also contain a sewing ring 336. The first part 330 bends laterally above the intersection with the second part 332 in this embodiment. FIGS. 5I and 5J show yet another stent structure 344 including a first part 340 connected to a second part 342 with a sewing ring 346 disposed on the first part 340. A lower portion of the first part 340 may extend beyond the intersection of the first part 340 and the second part 342. The quonset-shaped second part 342 in this embodiment extends so that it is adapted to engage a vessel around substantially the full circumference of a vessel.

The described stent structure 36 may be self-expanding, balloon expandable, or may have both characteristics. For example, a zig-zag stent is a stent structure that has alternating struts and peaks (i.e., bends) and defines a generally cylindrical space. A “Gianturco Z stent” is a type of self-expanding zig-zag stent structure. Stent structures may be encapsulated, partially encapsulated, or not encapsulated. A variety of other stent structures and configurations are also contemplated by use of the term stent structure.

A stent structure 36 offers the advantage of providing desirable forces in a specific direction or directions. Desirable forces include resilient forces and radial forces. The forces provided by a stent structure 36 may, among other things, resist the collapse of tissue walls, maintain a desirable geometry, provide expansion force to hold a device in place, seal an aperture, or otherwise provide desirable effects. Furthermore, stent structures provide the advantage of being able to be inserted with a narrow profile through apertures such as the incision 24 shown in FIG. 1, and then expanded at a desirable location to have a larger profile.

The stent structures 36 described herein are preferably self-expanding and formed from a superelastic material. When stent structures 36 are comprised of superelastic material, they are capable of elastically expanding to a predictable shape and offer the advantage of being able to spring back from external forces. Typically, superelastic materials can achieve elastic strains of at least several percent. Upon removal of the applied stress, the elastic strain induced by the applied stress is recovered and the material returns to its original, undeformed configuration. One example of a superelastic material is NITINOL, which is a superelastic nickel-titanium alloy that can achieve an elastic strain of about 8%. In contrast, 3conventional metal alloys, such as 304 stainless steel, typically achieve elastic strains of only a fraction of a percent. Materials exhibiting superelastic behavior are sometimes referred to as shape memory materials or pseudoelastic materials.

Accordingly, the stent structures may comprise self-expanding struts. The self-expanding struts may be made out of stainless steel, superelastic materials such as NITINOL, or any other suitable material. The stent structure or stent structures may be formed from self-expanding stents such as Z-STENTS. Z-STENTS are available from Cook, Incorporated, Bloomington, Ind. USA.

In some embodiments, such as the embodiment shown in FIG. 1, the graft tube 14 is attached to the stent structure 36 with a sewing ring 32 and a suture 34. The incorporation of a sewing ring 32 provides a number of advantages. For example, a sewing ring 32 provides for quicker attachment versus traditional suturing involving multiple sutures. Although multiple sutures may be used to secure the second end portion to the graft tube, uniformity is increased with a sewing ring 32 because less skill is required to attach a sewing ring 32 quickly and effectively. The sewing ring 32 which is shown is only one example of a structure for attaching the graft tube 14 to the second end portion 16, and many other ways of attachment known in the art may also be used. For example, in other embodiments, the graft tube may be attached to the second end portion with traditional suturing, stapling, gluing, or other alternatives known in the art.

In some embodiments, the graft tube comprises an extracellular matrix material. The “extracellular matrix” is typically a collagen-rich substance that is found in between cells in animal tissue and serves as a structural element in tissues. Such an extracellular matrix is preferably a complex mixture of polysaccharides and proteins secreted by cells. The extracellular matrix can be isolated and treated in a variety of ways. Following isolation and treatment, it is referred to as an “extracellular matrix material,” or ECMM. ECMMs may be isolated from submucosa (including small intestine submucosa), stomach submucosa, urinary bladder submucosa, tissue mucosa, renal capsule, dura mater, liver basement membrane, pericardium or other tissues.

Purified tela submucosa, a preferred type of ECMM, has been previously described in U.S. Pat. Nos. 6,206,931, 6,358,284 and 6,666,892 as bio-compatible, non-thrombogenic material that enhances the repair of damaged or diseased host tissues. U.S. Pat. Nos. 6,206,931, 6,358,284 and 6,666,892 are incorporated herein by reference. Purified submucosa extracted from the small intestine (“small intestine submucosa” or “SIS”) is a more preferred type of ECMM for use in this invention. Another type of ECMM, isolated from liver basement membrane, is described in U.S. Pat. No. 6,379,710, which is incorporated herein by reference. ECMM may also be isolated from pericardium, as described in U.S. Patent No. 4,502,159, which is also incorporated herein by reference.

Extracellular matrix materials such as purified tela submucosa, are substantially biocompatible and thus cause a reduced foreign body response when implanted within a body. Biocompatibility represents a problem for certain anastomosis devices. Some implanted biomaterials used for tissue repair initiate a foreign body reaction that causes encapsulation of the anastomosis device or a portion of the anastomosis device in rigid, fibrous scar tissue. Accordingly, a graft material comprising a biocompatible material is preferred.

Certain anastomosis devices include members, such as gripping hooks or connector wires, which attach to a vessel via a mechanism that causes trauma to a vessel by penetrating the inner surface of a vessel. Penetrating members apply a force at specific narrow points of contact in a manner that may damage a vessel. With a severe type of penetrating, the vessel wall is actually punctured by the penetrating members. For example, a device of U.S. Pat. No. 6,451,048 as shown in FIG. 1 in the first end portion 12, has upper connector wires 22 and lower connector wires 21 that may secure an anastomosis device to a vessel by puncturing the vessel wall.

Hooks and other devices for securing devices to vessels may have negative health consequences. Members that cause vessel trauma, such as gripping hooks which penetrate the inner surface of a vessel wall, may cause inflammation of the surrounding tissue. Inflammation may lead to an immune response whereby the foreign structure is encapsulated by new tissue growth which then occludes blood flow. In addition, inflammation may lead to thromboses or embolism. Alternatively, the trauma caused to the vessel may lead to a deterioration of the tissue wall around the foreign material, sometimes referred to as tissue erosion. In contrast, structures that apply a force over a surface area instead of applying force at a specific point or points may cause less vessel trauma.

In some embodiments, and as shown in FIG. 4, the graft tube 222 is a harvested vessel. The harvested vessel may be cut and attached to the second end portion 16 prior to insertion of the second end portion 16 through the incision 24. Alternatively, the harvested vessel may be attached to the second end portion 16 after the second end portion 16 is inserted through the incision 24.

The incision 24 may be made in a variety of ways. For example, an incision 24 may be made from the outside of the vessel, such as is typically done with a scalpel or a punch device. For further example, the incision 24 may also be made from the inside of the vessel, such as with a blade that may be advanced vascularly within a catheter.

In other embodiments, the graft tube comprises a biocompatible polyurethane. Examples of biocompatible polyurethanes include THORALON® (Thoratec, Pleasanton, Calif.), BIOSPAN®, BIONATE®, ELASTHANE™, PURSIL™ and CARSOSIL™ (Polymer Technology Group, Berkeley, Calif.). As described in U.S. Patent Application Publication No. 2002/006552 A2, incorporated herein by reference, THORALON® is a polyetherurethane urea blended with a siloxane-containing surface modifying additive. Specifically, the polymer is a mixture of base polymer BPS-215 with an additive SMA-300.

THORALON® has been used in certain vascular applications and is characterized by thromboresistance, high tensile strength, low water absorption, low critical surface tension, and good flex life. THORALON® is believed to be biostable and to be useful in vivo in long term blood contacting applications requiring biostability and leak resistance. Because of its flexibility, THORALON® is useful in procedures involving larger vessels where elasticity and compliance is beneficial.

Biocompatible polyurethanes modified with cationic, anionic and aliphatic side chains may also be used. See, for example, U.S. Pat. No. 5,017,664. Other biocompatible polyurethanes include: segmented polyurethanes, such as BIOSPAN; polycarbonate urethanes, such as BIONATE; and polyetherurethanes such as ELASTHANE; (all available from POLYMER TECHNOLOGY GROUP, Berkeley, Calif.).

Other biocompatible polyurethanes include polyurethanes having siloxane segments, also referred to as a siloxane-polyurethane. Examples of polyurethanes containing siloxane segments include polyether siloxane-polyurethanes, polycarbonate siloxane-polyurethanes, and siloxane-polyurethane ureas. Specifically, examples of siloxane-polyurethane include polymers such as ELAST-EON 2 and ELAST-EON 3 (AORTECH BIOMATERIALS, Victoria, Australia); polytetramethyleneoxide (PTMO) and polydimethylsiloxane (PDMS) polyether-based aromatic siloxane-polyurethanes such as PURSIL-10, -20, and -40 TSPU; PTMO and PDMS polyether-based aliphatic siloxane-polyurethanes such as PURSIL AL-5 and AL-10 TSPU; aliphatic, hydroxy-terminated polycarbonate and PDMS polycarbonate-based siloxane-polyurethanes such as CARBOSIL-10, -20, and -40 TSPU (all available from POLYMER TECHNOLOGY GROUP). The PURSIL, PURSIL -AL, and CARBOSIL polymers are thermoplastic elastomer urethane copolymers containing siloxane in the soft segment, and the percent siloxane in the copolymer is referred to in the grade name. For example, PURSIL-10 contains 10% siloxane. These polymers are synthesized through a multi-step bulk synthesis in which PDMS is incorporated into the polymer soft segment with PTMO (PURSIL) or an aliphatic hydroxy-terminated polycarbonate (CARBOSIL). The hard segment consists of the reaction product of an aromatic diisocyanate, MDI, with a low molecular weight glycol chain extender. In the case of PURSIL-AL the hard segment is synthesized from an aliphatic diisocyanate. The polymer chains are then terminated with a siloxane or other surface modifying end group. Siloxane-polyurethanes typically have a relatively low glass transition temperature, which provides for polymeric materials having increased flexibility relative to many conventional materials. In addition, the siloxane-polyurethane can exhibit high hydrolytic and oxidative stability, including improved resistance to environmental stress cracking. Examples of siloxane-polyurethanes are disclosed in U.S. Pat. Application Publication No. 2002/0187288 A1, which is incorporated herein by reference.

In addition, any of these biocompatible polyurethanes may be end-capped with surface active end groups, such as, for example, polydimethylsiloxane, fluoropolymers, polyolefin, polyethylene oxide, or other suitable groups. See, for example the surface active end groups disclosed in U.S. Pat. No. 5,589,563, which is incorporated herein by reference.

In other embodiments, the graft tube comprises DACRON, expanded polytetrafluoroethylene, or other suitable graft materials.

While preferred embodiments of the invention have been described, it should be understood that the invention is not so limited, and modifications may be made without departing from the invention. The scope of the invention is defined by the appended claims, and all devices and methods that come within the meaning of the claims, either literally or by equivalence, are intended to be embraced therein. Furthermore, the advantages described above are not necessarily the only advantages of the invention, and it is not necessarily expected that all of the described advantages will be achieved with all embodiments of the invention.

I claim:

1. An anastomosis device for bypassing a vessel, comprising:
a graft tube comprising a first end, a second end, and a lumen extending therethrough;
a first end portion connected to said first end of said graft tube, said first end portion being adapted to be attached to an incision in a vessel;
a second end portion connected to said second end of said graft tube, said second end portion comprising a stent structure, and said second end portion being adapted to be attached to another incision in said vessel;
wherein said stent structure comprises a first part adapted to be connected to said second end of said graft tube, said first part being substantially cylindrical, and a second part connected to said first part and being substantially quonset-shaped and adapted to engage a wall of said vessel, wherein said first part is connected to a convex region of said second part.

2. The anastomosis device according to claim 1, wherein said graft tube further comprises an extracellular matrix material.

3. The anastomosis device according to claim 1, wherein said graft tube comprises a biocompatible polyurethane.

4. The anastomosis device according to claim 1, wherein said second end portion comprises a biocompatible polyurethane that encapsulates said stent structure.

5. The anastomosis device according to claim 1, wherein said first part of said second end portion comprises a sewing ring incorporated into said first part of said second end portion.

6. The anastomosis device according to claim 1, wherein said first end portion comprises another stent structure comprising a first part adapted to be connected to said first end of said graft tube, said first part being substantially cylindrical, and a second part being substantially quonset-shaped and adapted to engage a wall of said vessel, wherein said first part is connected to a convex region of said second part.

7. The anastomosis device according to claim 6, wherein said first part of said another stent structure comprises a sewing ring incorporated into said first part of said first end portion.

8. An anastomosis device for bypassing a vessel, comprising:
a graft tube comprising a first end, a second end, and a lumen extending therethrough;
a first end portion connected to said first end of said graft tube, said first end portion being adapted to be attached to an incision in a vessel;
a second end portion connected to said second end of said graft tube, said second end portion comprising a stent structure, and said second end portion being adapted to be attached to another incision in said vessel;
wherein said stent structure comprises a first part adapted to be connected to said second end of said graft tube, said first part being substantially cylindrical, and a second part connected to said first part and adapted to engage a wall of said vessel, said second part extending generally laterally from said first part, wherein said stent structure engages said wall of said vessel without members attaching to said vessel by penetrating an inner surface of said vessel.

9. The anastomosis device according to claim 8, wherein said graft tube further comprises an extracellular matrix material.

10. The anastomosis device according to claim 8, wherein said graft tube comprises a biocompatible polyurethane.

11. The anastomosis device according to claim 8, wherein said second end portion comprises a biocompatible polyurethane that encapsulates said stent structure.

12. The anastomosis device according to claim 8, wherein said first part of said second end portion comprises a sewing ring incorporated into said first part of said second end portion.

13. The anastomosis device according to claim 8, wherein said stent structure comprises a first part adapted to be connected to said second end of said graft tube, said first part being substantially cylindrical, and a second part being substantially quonset-shaped, wherein said first part is connected to a convex region of said second part.

14. The anastomosis device according to claim 8, wherein said first end portion comprises another stent structure comprising a first part adapted to be connected to said first end of said graft tube, said first part being substantially cylindrical, and a second part being substantially quonset-shaped, wherein said first part is connected to the convex region of said second part.

15. The anastomosis device according to claim 14, wherein said first part of said first end portion comprises a sewing ring incorporated into said first part of said first end portion.

16. A method of using an anastomosis device comprising:
creating a first incision in a vessel;
attaching a first end portion of an anastomosis device at the location of the first incision;
creating a second incision in a vessel;
inserting a separate compressed second end portion of the anastomosis device through the second incision;
expanding the second end portion into a generally relaxed state; and
attaching a graft tube of the anastomosis device to the second end portion.

17. The method of claim 16, wherein the second end portion is disposed within an inner lumen of a retention sheath prior to expanding the second end portion into the generally relaxed state and the second end portion exits the retention sheath to expand.

18. The method of claim 17, wherein the second end portion is attached to the graft tube after the second end portion is expanded within the second incision.

19. The method of claim 18, wherein the second end portion is attached to the graft tube by wrapping a suture around a sewing ring disposed on the second end portion.

20. The method of claim 17, wherein the second end portion is fixedly attached to said graft tube prior to inserting said second end portion through the second incision.

21. The method of claim 17, wherein said first end portion is inserted through the first incision in a compressed state, said first end portion being fixedly attached to said graft tube prior to inserting said first end portion.
